(19) United States
(12) Patent Application Publication
(10) Pub. No.: US 2010/0241113 A1
(43) Pub. Date: Sep. 23, 2010

(54) PROTECTING THE PHRENIC NERVE WHILE ABLATING CARDIAC TISSUE

(75) Inventor: Frank Ingle, Palo Alto, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: Boston Scientific Scimed, Inc., Maple Grove, MN (US)

(21) Appl. No.: 12/727,580

(22) Filed: Mar. 19, 2010

Related U.S. Application Data
(60) Provisional application No. 61/161,968, filed on Mar. 20, 2009.

(51) Int. Cl. A61B 18/02

(52) U.S. Cl. 606/21

(57) ABSTRACT

In some implementations, a cryotherapy delivery system includes a cryotherapy catheter having a distal treatment component that delivers, during a cryotherapy procedure, cryotherapy to a treatment site inside a patient's body; a controller that controls the delivery of the cryotherapy during the cryotherapy procedure; and a sensor that measures values of a respiration parameter of the patient during the cryotherapy procedure, and provides measured values to the controller. The controller can determine, prior to delivery of cryotherapy, a baseline value for the respiration parameter; detect, during delivery of the cryotherapy, a change in the respiration parameter relative to the baseline value; and suspend delivery of the cryotherapy when the change exceeds a threshold.

### CROSS-REFERENCE TO RELATED APPLICATION

This application claims priority under 35 USC §119(e) to U.S. Provisional Patent Application Ser. No. 61/161,968, filed on Mar. 20, 2009, the entire contents of which are hereby incorporated by reference.

### BACKGROUND

A number of serious medical conditions may be treated in a minimally invasive manner with various kinds of catheters designed to reach treatment sites internal to a patient's body. One such medical condition is atrial fibrillation—a condition that results from abnormal electrical activity within the heart. This abnormal electrical activity may originate from various focal centers of the heart and generally decreases the efficiency with which the heart pumps blood. It is believed that some of these focal centers reside in the pulmonary veins of the left atrium. It is further believed that atrial fibrillation can be reduced or controlled by structurally altering or ablating the tissue at or near the focal centers of the abnormal electrical activity, such that the ablated tissue is electrically isolated from surrounding tissue.

One method of ablating tissue of the heart and pulmonary veins to treat atrial fibrillation is cryotherapy—the extreme cooling of body tissue. Cryotherapy may be delivered to appropriate treatment sites inside a patient's heart and circulatory system by a cryotherapy catheter. A cryotherapy catheter generally includes a treatment member at its distal end, such as a metal tip or an expandable balloon having a cooling chamber inside. A cryogenic fluid may be provided by a source external to the patient at the proximal end of the cryotherapy catheter and delivered distally through a lumen to the cooling chamber where it is released. Release of the cryogenic fluid into the chamber cools the chamber (e.g., through evaporation of the fluid), and correspondingly, the balloon's outer surface, which is in contact with tissue that is to be ablated. Gas resulting from evaporation of the cryogenic fluid may be exhausted proximally through an exhaust lumen to a reservoir or pump external to the patient. Another method of ablating tissue of the heart and pulmonary veins to treat atrial fibrillation involves delivering radio-frequency (RF) energy to tissue.

### SUMMARY

A cryotherapy system for electrically isolating a patient's pulmonary veins (e.g., to treat atrial fibrillation) can monitor a respiration parameter of the patient and automatically suspend delivery of the cryotherapy when a change in the respiration parameter is detected that indicates a risk of imminent nerve damage to the patient. Such a system can reduce the risk of damage to the patient's right phrenic nerve, which controls the function of the right side of the diaphragm and is typically located close to one of the patient's pulmonary veins.

In some implementations, a cryotherapy delivery system includes a cryotherapy catheter having a distal treatment component that delivers, during a cryotherapy procedure, cryotherapy to a treatment site inside a patient's body; a controller that controls the delivery of the cryotherapy during the cryotherapy procedure; and a sensor that measures values of a respiration parameter of the patient during the cryotherapy procedure, and provides measured values to the controller. The controller can determine, prior to delivery of cryotherapy, a baseline value for the respiration parameter; detect, during delivery of the cryotherapy, a change in the respiration parameter relative to the baseline value; and suspend delivery of the cryotherapy when the change exceeds a threshold.

The controller can control the delivery of the cryotherapy by regulating the flow of a cryogenic agent to and from the distal treatment component to control a temperature or pressure of the treatment component. The controller can provide an alarm signal when the change exceeds a warning threshold that is smaller than the threshold for damage. In some implementations, the warning threshold is 10%. In some implementations, the threshold is 25%. The sensor can include a respiration sensor, such as, for example, an extensiometer that measures expansion and contraction of the patient's chest or abdomen, or an impedance plethysmograph that measures changes in chest impedance. The sensor can include a flow monitor that measures an inspiratory flow rate or expiratory flow rate. The sensor can include a pulse oximeter that measure an oxygen saturation value of the patient's blood. The treatment component can include an expandable balloon.

In some implementations, a method of providing cryotherapy includes introducing a cryotherapy catheter at a treatment site inside a patient's heart and determining a baseline value for a respiration parameter of the patient. The method can further include employing an electronic controller of the cryotherapy catheter to regulate delivery of cryotherapy to the treatment site. While cryotherapy is being delivered to the treatment site, the method can include detecting a change in the respiration parameter, relative to the baseline value, that exceeds a threshold, and in response to detecting the change, the electronic controller can alert a physician or automatically suspend delivery of the cryotherapy.

In some implementations, the threshold includes at least one of 10%, 25% or 50% of the average baseline value. The treatment site can be the antrum of a pulmonary vein of the patient. Detecting a change that exceeds the threshold can include detecting a change in function of the patient's diaphragm that is indicative of reversible (e.g., transient) paralysis of the patient's phrenic nerve. Determining the baseline and detecting the change can include receiving values from a sensor that is coupled to the electronic controller. The sensor can include an extensiometer that measures expansion and contraction of the patient's chest or abdomen. The sensor can include a flow monitor that measure an inspiratory flow rate or expiratory flow rate. The sensor can include a pulse oximeter that measure an oxygen saturation value of the patient's blood. A method of providing cryotherapy can further include supplying heat to a region of the patient's esophagus that is in close proximity to the treatment site.

The details of one or more implementations are set forth in the accompanying drawings and the description below. Other features, objects, and advantages will be apparent from the description and drawings, and from the claims.

### BRIEF DESCRIPTION OF DRAWINGS

FIG. 1 is a diagram of a cryotherapy system that can automatically control delivery of cryotherapy in response to monitored respiration parameters of a patient.

FIG. 2 depicts the cryotherapy system of FIG. 1 as it may be employed during a cryotherapy procedure.

FIGS. 3A to 3E depict a cold front propagating from a treatment component of the cryotherapy system of FIG. 1, during a procedure, such as the one depicted in FIG. 2.

FIG. 4 illustrates the anatomical relationship between different body tissues and structures that may be affected in a procedure such as the one depicted in FIG. 2.

FIG. 5 is a flow diagram of an example method of providing cryotherapy.

Like reference symbols in the various drawings indicate like elements.

### DETAILED DESCRIPTION

A cryotherapy system for electrically isolating a patient's pulmonary veins (e.g., to treat atrial fibrillation) can monitor a respiration parameter of the patient and alert a physician or automatically suspend delivery of the cryotherapy when a change in the respiration parameter is detected that indicates a risk of imminent nerve damage to the patient. Such a system can reduce the risk of damage to the patient's right phrenic nerve, which controls the function of the right side of the diaphragm and is typically located close to the right superior pulmonary vein—one of four pulmonary veins that are typically ablated in cryotherapy procedures for treating atrial fibrillation. If cryotherapy is delivered for too long of a period of time to this pulmonary vein in particular, a cold front can propagate through the walls of the pulmonary vein and impinge upon the phrenic nerve. If the temperature of the cold front is too cold, or if the cold front impinges upon the phrenic nerve for too long of a period of time, the nerve may be permanently damaged.

Prior to permanently damaging the nerve, the cold front may temporarily and reversibly reduce the phrenic nerve's ability to transmit signals, which can cause transient paralysis of a portion of the diaphragm. By electronically monitoring a respiration parameter of the patient during a cryotherapy procedure, and comparing the monitored respiration parameter to a baseline established prior to the procedure, the system can automatically detect the transient paralysis and suspend delivery of cryotherapy before the phrenic nerve is permanently damaged.

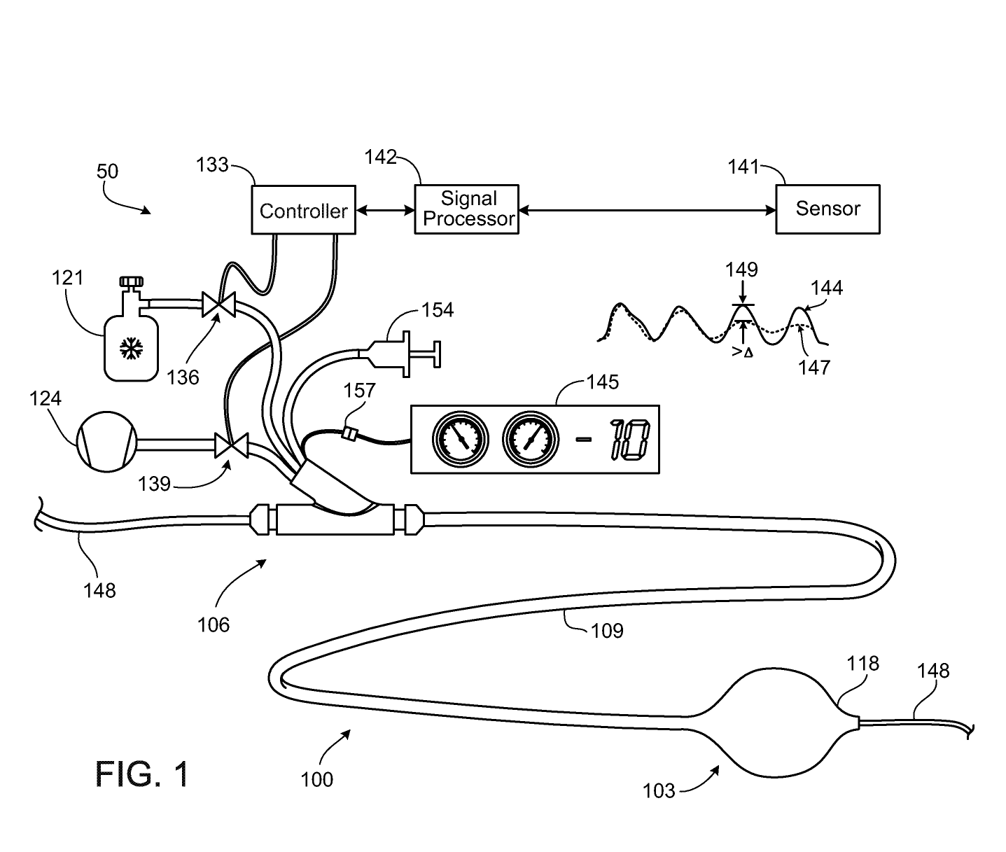

FIG. 1 is a diagram of a cryotherapy system 50 that can automatically control delivery of cryotherapy in response to a monitored respiration parameter. As shown in one implementation, the cryotherapy catheter 100 includes a distal inflatable balloon portion 103 that can be routed to a treatment site inside a patient to deliver cryotherapy to that treatment site; a proximal end 106 that remains outside the patient during treatment and facilitates connection of various equipment to the cryotherapy catheter; and an elongate member 109 that couples the proximal-end equipment to the distal inflatable balloon portion. In other implementations (not shown), other distal treatment components, such as a hollow metal tip, may be employed in place of the example inflatable balloon portion 103.

The catheter's elongate member 109 can have multiple internal lumens (not shown) that allow cryogenic fluid to be delivered distally from an external cryogenic fluid source 121 to an internal chamber of the balloon 103. In addition, the internal lumens of the elongate member 109 allow exhaust resulting from delivery of cryogenic fluid to the internal chamber of the balloon 103 to be delivered proximally from the internal chamber to, for example, an external exhaust pump 124. During operation, there may be continuous circulation within the elongate member 109 of cryogenic fluid distally and exhaust proximally.

A controller 133 can regulate flow of cryogenic fluid to the internal chamber of the balloon 103 and flow of exhaust from the balloon 103. In particular, for example, the controller 133 can, in one implementation as shown, regulate a valve 136 that controls flow of the cryogenic fluid from the cryogenic fluid source 121. The cryogenic fluid source 121 may be, for example, a pressured flask of cryogenic fluid. In other implementations (not shown), the controller controls a pump or pump/valve combination to deliver cryogenic fluid to the internal chamber of the balloon. Similarly, the controller 133 can regulate a valve 139 and/or external exhaust pump 124 to regulate flow of exhaust from the internal chamber of the balloon.

By controlling both the rate at which cryogenic fluid is delivered to the balloon 103 and the rate at which exhaust is extracted from the balloon 103, the controller 133 can regulate the pressure inside the balloon 103 and cause the surface 118 of the balloon 103 to have a desired temperature (or more precisely, the controller can control an amount of heat to be extracted from the surface 118 and from body tissue that is in contact with the surface 118). For example, when cryogenic fluid is delivered at a very low rate to the balloon 103, and exhaust is similarly extracted at a very low rate, very little heat (if any) may be extracted from the balloon 103 or from body tissue that is in contact with the balloon's surface 118; the flow may merely keep the balloon 103 inflated. As another example, when cryogenic fluid is delivered at a higher rate, a large amount of heat can be extracted from the balloon 103 and from body tissue that is in contact with the balloon 103, such that the adjacent tissue is cryo-ablated.

To precisely control flow rates, the controller 133 may employ either or both of open- or closed-loop control systems. For example, in some implementations, a rate at which cryogenic fluid (e.g., the position of the valve 136) may be controlled with an open-loop control system, and a rate at which exhaust is extracted from the balloon 103 (e.g., the position of the valve 139, or the pressure exerted by the pump 124) may be controlled with a closed-loop control system. In other implementations, both rates may be controlled by closed-loop control systems. In a closed-loop control system, some feedback mechanism is provided. For example, to control the rate at which exhaust is extracted from the balloon 103, the controller 133 may employ an exhaust flow sensor device (not shown), a pressure sensor (not shown) inside the balloon 103 or elsewhere in the system, or another feedback sensor (e.g., a temperature sensor).

The control system can also receive input that can be used to gate delivery of cryotherapy. For example, the control system can receive input associated with a respiration parameter of the patient receiving cryotherapy. As long as the respiration parameter is within a normal range (e.g., within a threshold amount or percentage of a baseline value), cryotherapy can be delivered in a controlled manner as described above; if a change that exceeds a predetermined threshold is detected in the respiration parameter of the patient, delivery of cryotherapy can be automatically suspended, or a warning signal can be provided.

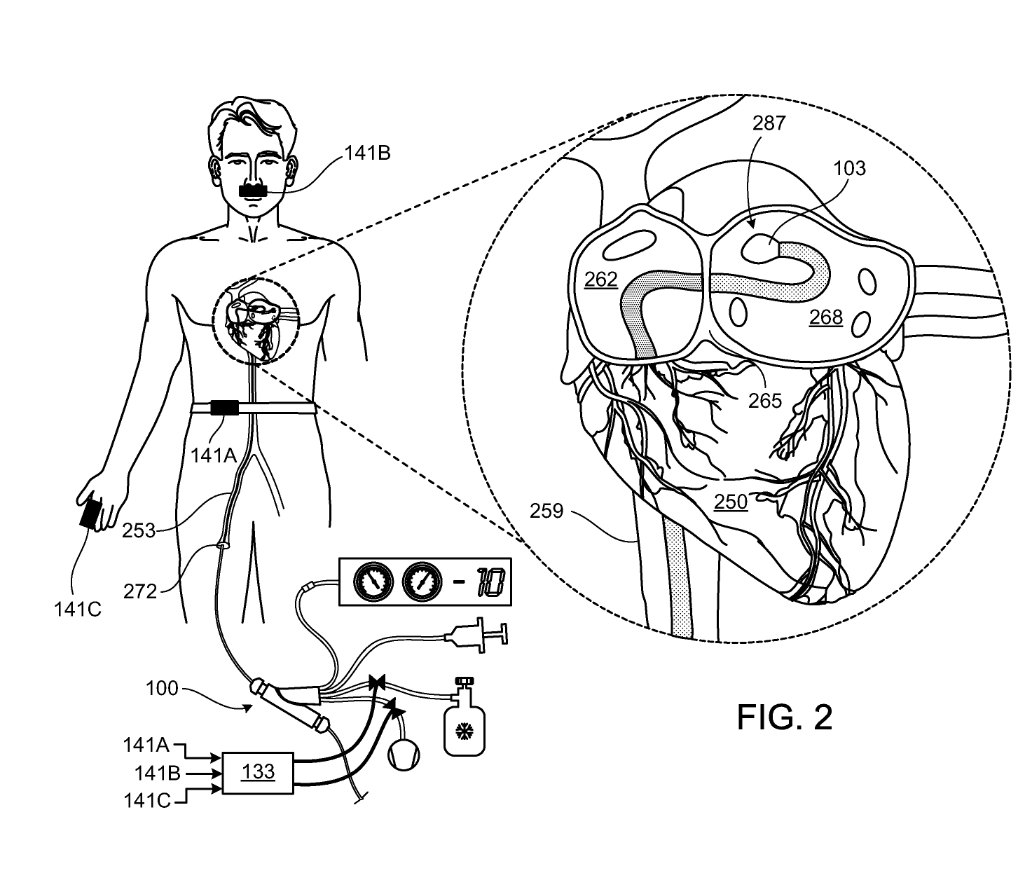

In some implementations, the control system receives respiration input from a respiration sensor 141 that is coupled to the patient. Various kinds of respiration sensors can be employed. For example, with reference to FIG. 2, an extensiometer 141A, such as an elastic band that measures an extent to which the band is stretched, can be placed around a patient's chest or abdomen to measure chest displacement associated with breathing. As another example, a flow sensor 141B can be placed in or inline with a patient's nasal or oral airway, to measure, for example, inspiratory and/or expiratory flow rate or pressure. As another example, a pulse oximeter 141C can be employed to measure oxygen saturation in the patient's blood, which generally corresponds to the patient's breathing patterns and breathing quality.

The preceding examples are not exhaustive, and the reader will appreciate that numerous other sensors can be employed to measure parameters associated with a patient's breathing. In general, any sensor can be employed whose data would facilitate a determination of a change in breathing quality (e.g., a reduction in tidal volume, a reduction in flow rate or pressure, a reduction in the amount of oxygen absorbed in the blood, etc.) that may be associated with a change in diaphragm function, which may, in turn, indicate a change in phrenic nerve function.

Regardless of the specific sensor employed, data from the sensor can be provided to the controller 133 for use in gating delivery of cryotherapy. In particular, with continued reference to FIG. 1, the controller 133 can use data gathered before cryotherapy is delivered to determine a baseline 144 value for the respiration parameter being measured (e.g., tidal volume, breathing rate, flow rate or pressure, absorbed oxygen, etc.). During delivery of the cryotherapy, the controller 133 can analyze data 147 from the sensor in real-time to detect any changes in the respiration parameter relative to the baseline. If the change (e.g., the change 149) exceeds a predetermined threshold, the controller 133 can suspend delivery of cryotherapy. For example, upon detecting such a change, the controller 133 could close the valve 136 to stop or reduce the flow of cryogenic fluid to the balloon 103. In some cases, suspending delivery of cryotherapy at such times can protect the patient against damage to the phrenic nerve.

To analyze data from the sensor, the controller 133 can employ various signal processing techniques and systems. For example, the controller 133 can determine and track a per-cycle or average peak amplitude of a respiration parameter signal before cryotherapy is delivered. During delivery of the cryotherapy, the controller 133 can determine a per-cycle peak amplitude of the same parameter and directly compare the per-cycle peak amplitude or a running average of recent per-cycle peak amplitudes to a baseline value. More specifically, as depicted in FIG. 1, the controller 133 may be able to determine a point 149 at which the per-cycle peak amplitude is more than a predetermined threshold (e.g., Δ) different from the baseline. The controller may analyze data from multiple sensors in gating delivery of cryotherapy or in providing physician alerts. For example, the controller can analyze tidal volume and oxygenation and gate delivery of cryotherapy or generate an alert when tidal volume increases more than a predetermined amount and blood oxygenation decreases by more than a second predetermined amount.

To determine per-cycle peak values, the controller 133 may calculate a derivative (i.e., slope) of a respiration signal, and use the derivative to determine peaks or troughs in the signal. Such peaks or troughs may be helpful in aligning a real-time respiration signal to a previously measured baseline signal. In other cases, the derivative itself may be used for establishing a baseline and subsequent comparison to the baseline. In particular, for example, a derivative of air flow may be employed to determine a flow rate, and the flow rate may be subsequently analyzed. In still other cases, a respiration signal may be integrated and the integral may be used for subsequent analysis. In particular, for example, a flow rate signal may be integrated to determine a volume of air (e.g., a tidal volume for a portion of an inspiration or expiration cycle).

In implementations in which a derivative or an integral of a respiration signal is analyzed, the signal may be analyzed in substantially real-time. That is, the signal may be analyzed promptly (e.g., within one or two respiration cycles), but the inherent processing associated with calculating a derivative or integral may necessarily require a certain number of data points. More specifically, detecting with certainty a peak in a respiration signal may require that a negative slope be detected for a threshold period of time; thus, to precisely identify the peak, the controller may need to receive data points that follow the peak. Similarly, to determine a tidal volume by integrating a flow rate, the volume for the preceding cycle may not be available until data points for the entire cycle have been received. Thus, in some scenarios, respiration parameters may be processed in substantially real-time, with some small amount of delay.

In some implementations, a signal processor 142 that is separate from the controller 133 can be employed. For example, a separate signal processor 142 may be included for interfacing to the respiration sensor(s); sampling sensor output; calculating derivatives, integrals or performing other manipulations of the data; comparing baseline data with real-time (or substantially real-time data); etc. In such implementations, an output of the signal processor 142 may serve as a gating signal that either allows cryotherapy to be delivered according to other control parameters, or prevents or suspends delivery of cryotherapy. In other implementations, the signal processor 142 is omitted, and the sensor 141 is coupled directly to the controller 133.

The controller 133 itself can take many different forms. In some implementations, the controller 133 is a dedicated electrical circuit employing various sensors, logic elements, and actuators. In other implementations, the controller 133 is a computer-based system that includes a programmable element, such as a microcontroller or microprocessor, which can execute program instructions stored in a corresponding memory or memories. Such a computer-based system can take many forms, include many input and output devices (e.g., a user interface and other common input and output devices associated with a computing system, such as keyboards, point devices, touch screens, discrete switches and controls, printers, network connections, indicator lights, etc.) and may be integrated with other system functions, such as monitoring equipment 145 (described in more detail below), a computer network, other devices that are typically employed during a cryotherapy procedure, etc. For example, a single computer-based system may include a processor that executes instructions to provide the controller function, display imaging information associated with a cryotherapy procedure (e.g., from an imaging device); display pressure, temperature and time information (e.g., elapsed time since a given phase of treatment was started); and serve as an overall interface to the cryotherapy catheter.

In general, various types of controllers are possible and contemplated, and any suitable controller 133 can be employed. Moreover, in some implementations, the controller 133 and the signal processor 142 may be part of a single computer-based system, and both control and signal processing functions may be provided, at least in part, by the execution of program instructions in a single computer-based system.

The catheter 100 shown in FIG. 1 may be an over-the-wire type catheter. Such a catheter 100 may use a guidewire 148, extending from the distal end of the catheter 100. In some implementations, the guidewire 148 may be pre-positioned inside a patient's body, and once the guidewire 148 is properly positioned, the balloon 103 (in a deflated state) and the elongate member 109 can be routed over the guidewire 148 to a treatment site. In some implementations, the guidewire 148 and balloon portion 103 of the catheter 100 may be advanced together to a treatment site inside a patient's body, with the guidewire portion 148 leading the balloon 103 by some distance (e.g., several inches). When the guidewire portion 148 reaches the treatment site, the balloon 103 may then be advanced over the guidewire 148 until it also reaches the treatment site. Other implementations are contemplated, such as steerable catheters that do not employ a guidewire. Moreover, some implementations include an introducer sheath that can function similar to a guidewire, and in particular, that can be initially advanced to a target site, after which other catheter portions can be advanced through the introducer sheath.

The catheter 100 can include a manipulator (not shown), by which a medical practitioner may navigate the guidewire 148 and/or balloon 103 through a patient's body to a treatment site. In some implementations, release of cryogenic fluid into the cooling chamber may inflate the balloon 103 to a shape similar to that shown in FIG. 1. In other implementations, a pressure source 154 may be used to inflate the balloon 103 independently of the release of cryogenic fluid into the internal chamber of the balloon 103. The pressure source 154 may also be used to inflate an anchor member on the end of the guidewire 148 (not shown).

The catheter 100 may include a connector 157 for connecting monitoring equipment 145. The monitoring equipment may be used, for example, to monitor temperature or pressure at the distal end of the catheter 100. The monitoring equipment can also be integrated with the controller 133 or a signal processor 142, to display information about the baseline or real-time respiration signal. For example, the monitoring equipment may display a baseline respiration signal (e.g., signal 144), and superimposed on the baseline signal a real-time, or substantially real-time respiration signal (e.g., signal 147) for comparison. The monitoring equipment may also include an indicator or alarm for alerting an operator of a change in the respiration parameter. More specifically, the monitoring equipment can, in some implementations, display baseline and substantially real-time respiration information, provide an audible or visual alarm when any significant change in the respiration parameter is detected, and provide a second audible or visual alarm when the change exceeds the predetermined threshold, such that delivery of cryotherapy has been suspended. As indicated above, the monitoring equipment 145 may be integrated in a single system that also provides the controller 133 and signal processor 142.

Other variations in the catheter 100 are contemplated. For example, the monitoring equipment 145 is shown separately in FIG. 1, but in some implementations, displays associated with the monitoring equipment are included in a single user interface (not shown). The controller 133 is depicted as controlling valves 136 and 139 to regulate the flow of cryogenic fluid to the balloon 103 and channeling exhaust from the balloon 103, but other control schemes (e.g., other valves or pumps) can also be employed. A guidewire 148 may be arranged differently than shown, and may be separately controlled from the balloon portion of the catheter. Moreover, in some implementations, a guidewire may not be used. Various kinds of respiration sensors can be employed. A dedicated signal processing component 142 can be included or omitted.

FIG. 2 is a diagram depicting a cryotherapy procedure in which the cryotherapy system 100 of FIG. 1 can be employed. In this example, the catheter 100 may deliver cryotherapy to the left atrium 268 of a patient's heart 250 in order to treat atrial fibrillation. By way of background, and for context, a medical practitioner may route the catheter 100 to the patient's left atrium 268 by accessing the patient's circulatory system at the patient's femoral vein 253. In particular, the medical practitioner may insert a sleeve or sheath 272 into the patient's femoral vein 253 to keep an access point open during the procedure. In some procedures, the medical practitioner advances a guidewire through the sheath 272, into the femoral vein 253 in the patient's upper leg, into the inferior vena cava 259, and into the patient's right atrium 262. In other procedures, the medical practitioner routes a delivery sheath (e.g., a steerable delivery sheath) along a similar path, and uses the delivery sheath 272 to subsequently route a guidewire-less catheter to a treatment site.

The medical practitioner may then puncture the septum 265. In particular, the medical practitioner may route a transseptal needle (not shown) over a guidewire or through a delivery sheath, puncture the septum with the transseptal needle to create an access point, withdraw the transseptal needle, then advance the guidewire or delivery sheath through the access point into the patient's left atrium 268. Once the guidewire or delivery sheath is in the patient's left atrium 268, the medical practitioner may advance the cryo balloon 103 portion of the catheter 100 to just outside one of the pulmonary veins (e.g., to the ostium of the pulmonary vein). In some implementations, the medical practitioner may then inflate the cryo balloon 103 such that its exterior surface contacts tissue at the circumference of the ostium; then the medical practitioner may initiate one or more cooling cycles to ablate the tissue of the ostium.

Once the tissue of one ostium 287 has been treated, the catheter 100 may be repositioned to treat other ostia. To reposition the catheter 100, the cryo balloon 103 may be deflated and the catheter 100 withdrawn enough to permit the guidewire or delivery sheath to be repositioned in or near another ostium. After the cryo balloon 103 is appropriately positioned, one or more cooling cycles may be initiated to ablate the tissue of this ostium. This process may be repeated for the other ostia, such that annular conduction blocks are created in multiple ostia. Once the entire therapy process has been completed, the cryo balloon 103 may again be deflated, and the catheter 100 may be removed from the patient. Similarly, the guidewire 148 may be removed.

Although the example procedure described above is largely in the context of a catheter having a guidewire, the procedure of ablating tissue with a cryo balloon catheter may also be performed with a fully steerable catheter that lacks a corresponding guidewire. Fully steerable, guidewire-less catheters are not described here in detail, as the exact structure of the steering mechanism of the catheter is not critical to this document; any appropriate steering mechanism may be used to advance the catheter to various treatment sites.

During each cooling cycle in the above-described example procedure, the delivery of cryotherapy can be controlled by the controller 133, based on input received from the sensor 141. That is, before any cryotherapy treatment cycle is initiated, a baseline can be established for a respiration parameter of the patient (e.g., after the catheter 100 is positioned, to allow the patient's respiration to settle out after possibly being affected by the procedure in which the catheter is routed to its treatment site); and during the procedure, additional data for the respiration parameter can be gathered and compared to the baseline. If the additional data indicates a change in the respiration parameter that exceeds a predetermined threshold, delivery of cryotherapy can be suspended. In this manner, patients whose phrenic nerves are located very close to pulmonary vein tissue can be protected against nerve damage during a cryotherapy procedure.

FIG. 2 further illustrates three example sensors that can be employed to provide to the controller 133 data corresponding to the patient's respiration function. In particular, an extensiometer 141A, for example one in the form of an elastic band that measures an extent to which the band is stretched, can be placed around a patient's chest or abdomen to measure chest displacement associated with breathing. The resistance of the band may change as it is stretched, and a resistance-time signal can be used to track timing an extent of chest or abdomen movement.

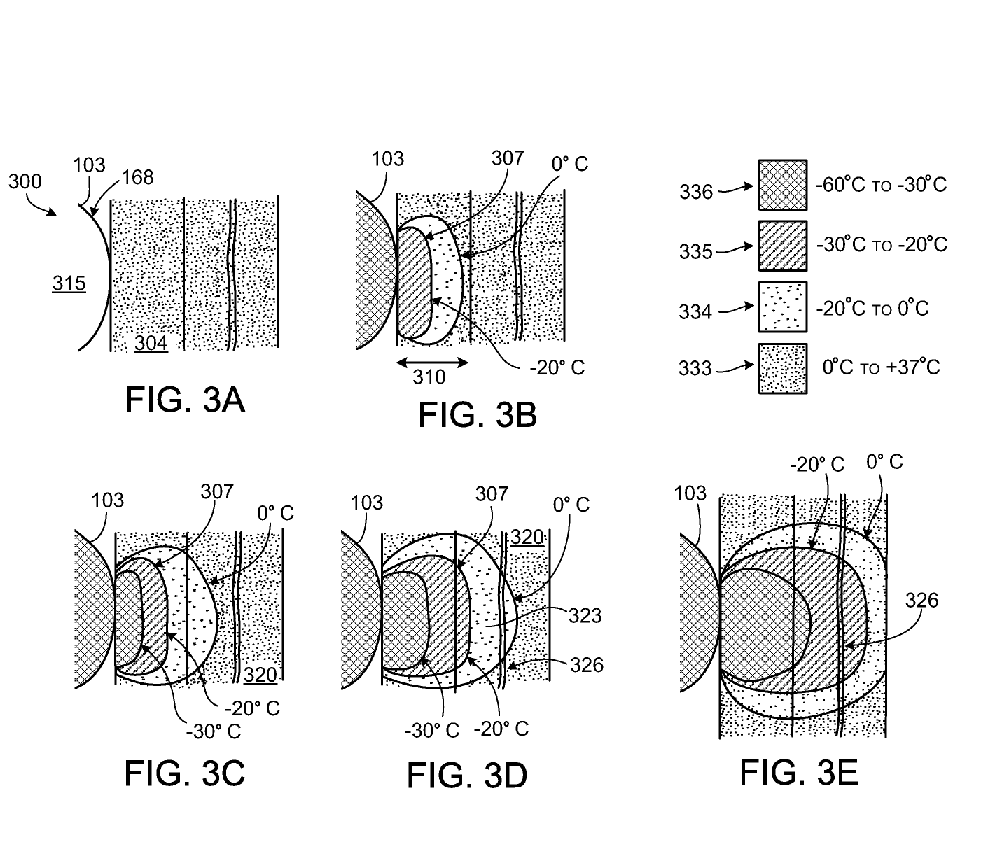

An average chest or abdomen displacement can be calculated from several breathing cycles and used as a baseline, before cryotherapy is delivered. Chest or abdomen movement can be monitored during delivery of cryotherapy (e.g., by monitoring a resistance-time signal from the extensiometer 141A), and the mid-procedure data can be compared to pre-procedure baseline data. If the mid-procedure data differs from the baseline data by more than a threshold amount (e.g., by more than 25% in some implementations), appropriate action can be taken (e.g., delivery of cryotherapy can be suspended). More specifically, changed respiration function (e.g., resulting from a transiently paralyzed diaphragm portion) can result in different chest or abdomen displacement relative to the baseline, which, in turn, can result in a different resistance-time signal. Thus, by detecting the different resistance-time signal, relative to the baseline, the controller 133 may be able to suspend delivery of cryotherapy in time to avoid nerve damage. Propagation of a cold front from the balloon 103 to a nerve, such as the phrenic nerve, is depicted in FIGS. 3A-3E and further described below.

Other sensors can be used to capture data that can be processed to identify points at which nerve damage may be imminent. Another example sensor is an airflow sensor 141B. The example airflow sensor 141B is depicted near a patient's nasal airway, but the airflow sensor can be disposed elsewhere. For example, in procedures in which the patient has a breathing tube in his or her mouth or throat, the airflow sensor can be disposed in or on the breathing tube. Wherever the airflow sensor is placed, it can, in some implementations, detect a flow rate or pressure associated with the patient's breathing. Like chest or abdomen displacement, a flow rate or pressure can serve as an indicator for overall breathing quality. Changes in flow rate or pressure between pre-procedure baseline data and mid-procedure data can indicate a reduction in breathing function, which may indicate that one of the phrenic nerves has been affected by the procedure.

Another example sensor is a pulse oximeter 141C. A pulse oximeter 141C can be employed to measure oxygen content of the blood, which is related to lung function (and thus indirectly related to diaphragm or phrenic nerve function). Thus, if the phrenic nerve is adversely affected by a procedure, the pulse oximeter 141C may provide oxygenation data from which the controller 133 can detect a decrease in respiration function. As described above, when a detected decrease in function exceeds a predetermined threshold, the controller 133 can suspend delivery of cryotherapy.

In some implementations in which a pulse oximeter is employed, the predetermined threshold may be lower than it would be for other types of sensors, since there may be more inherent delay between detection of an effect on the respiration parameter (e.g., detection of reduced oxygenation of the blood) and its cause (e.g., freezing of the phrenic nerve, causing reduced diaphragm function). In general, the threshold can be set to detect a change in respiration function while there is still time to suspend delivery of additional cryotherapy and prevent permanent damage to the phrenic nerve. In cases where physiological processes add delay to the detection (e.g., the process by which blood is oxygenated in the lungs and subsequently pumped to a location at which the pulse oximeter monitors the oxygenation level), the threshold can be set lower to partially compensate for the physiological delay.

Other sensors can employed. For example, in certain implementations, one or more sensors may be configured to detect the loss of right phrenic nerve function by monitoring the patient for increased chest expansion and more rapid respiration, which may be expected to result as the body naturally attempts to maintain the pressure of carbon dioxide (pCO₂) and pressure of oxygen (pO₂) at constant levels. Changes in these patient parameters may therefore reveal transient loss of phrenic function. In another example, some implementations may measure the electrical impedance of the chest (using, for example, an abdominal band electrode and a neck band electrode, or a back electrode and a front electrode) to sense the change in dimension or fraction of air contained in the chest and diaphragm area. In general, any sensor can be employed that gathers data associated with a respiration parameter, from which data change in respiration function can be detected that would be expected to result from transient paralysis of one of the phrenic nerves. In some implementations, multiple sensors can be employed in combination.

Propagation of a cold front through body tissue in a manner that can be detected by one of the above-described sensors is now described with reference to FIGS. 3A-E. FIGS. 3A to 3E depict a cold front propagating from a treatment component. For purposes of example, the treatment component is depicted as the balloon portion 103 of the cryotherapy catheter 100, which is illustrated in and described in greater detail with reference to FIG. 1. The reader will appreciate, however, that the principles describe herein can be applied to devices other than catheters. For simplicity, this description refers in various places to propagation of a cold front, but the reader will appreciate that propagation of a cold front may, more precisely, involve extraction of heat from progressively deeper tissue.

During a cryotherapy procedure, the balloon 103 can be positioned in contact with targeted tissue 304. For example, in a procedure to treat atrial fibrillation, the balloon 103 can be disposed inside a patient's heart, and more particularly, disposed at and against an ostium or antrum of one of the patient's pulmonary veins.

To deliver cryotherapy, a cryogenic agent can be delivered to a chamber 315 inside the balloon 103, in order to cool an outer surface 118 of the balloon 103 and, correspondingly, targeted body tissue 304 that is in contact with the outer surface 118. Cooling of the outer surface 118 causes a cold front to propagate into the targeted body tissue 304, as is depicted in and described with reference to FIGS. 3B-3E.

FIG. 3B depicts a cold front 307 that advances deeper into the body tissue 304 over time. As used herein, the cold front temperature can include a temperature that is therapeutically effective in treating (e.g., ablating) tissue. For example, some implementations involve cooling the outer surface 118 to about −60° C. or cooler, which creates a temperature gradient that includes the cold front 307 having a cold front temperature (e.g., about −20° C.) to advance into the body tissue 304. More generally, FIG. 3B depicts a temperature gradient that forms across a thickness 310 of the targeted body tissue 304 when the cooled outer surface 118 is in contact with the body tissue 304. FIGS. 3C and 3E illustrate the temperature gradient at later points in time, and further depict how the cold front 307 can penetrate deeper into the body tissue 304 over time.

As depicted, isotherms of varying temperature can be formed (e.g., loci of temperatures that spread into the tissue—in particular, temperatures within specific ranges, such as, for example −60° C. to −30° C., −30° C. to −20° C., −20° C. to 0° C., and 0° C. to 37° C.), example regions 333, 334, 335 and 336 of which are shown in FIGS. 3B-3E. For purposes of illustration, the granularity of the temperature range within each region 333-336 is quite large, but the reader will appreciate that an actual temperature gradient may have a range of temperatures that varies substantially continuously, or in smaller steps, rather than in the larger steps depicted.

As depicted in FIGS. 3C-3E, tissue 320 beyond the targeted body tissue 304 may also be cooled, depending on how deep the cold front 307—or more generally the temperature gradient—propagates into and beyond the targeted tissue 304. This depth can depend on various factors, including, for example, the type of targeted tissue 304; the thickness 310 of that tissue; other tissue, structures or spaces that are adjacent to the targeted tissue 304; physiology of the targeted tissue 304 and adjacent tissue 320 (e.g., a level of blood flow in either the targeted tissue or the adjacent tissue); and other factors.

In many procedures, it is advantageous to primarily limit the propagation of the cold front 307 (e.g., specifically, a cold front having a temperature that is less than or equal to about −20° C.) to the thickness 310 of the targeted tissue 304. That is, therapy may be most effective, and unintended and possibly adverse side effects may be prevented or minimized, if the cold front 307 propagates to a therapeutic depth (e.g., a significant fraction of the thickness 310) but does not propagate substantially beyond the thickness 310 of the targeted tissue 304. In this context, preventing of the cold front 307 from propagating substantially beyond the thickness 310 may include selecting a treatment time such that the cold front is not likely to propagate beyond the thickness 310 of the targeted body tissue by more than some percentage of the thickness 110 (e.g., 25%, 50%, 100%, 125%, etc.).

FIGS. 3A-3E illustrate another tissue structure 326 disposed beyond the targeted body tissue 304 and in or beyond the adjacent tissue 320. As a concrete example, the targeted body tissue 304 could be the vessel wall of a patient's pulmonary vein, the adjacent tissue 320 could be tissue of the pericardium, and the tissue structure 326 could be a nerve (e.g., the phrenic nerve) that is disposed close to the targeted tissue 304. Certain tissue, including nerve tissue, may be particularly susceptible to damage caused by heating or cooling. Thus, in this example, the nerve 326 may be irreversibly damaged if the cold front 307 were to impinge on it. More specifically, nerve tissue may be irreversibly damaged (e.g., killed) if exposed to temperatures at or below −20° C. Accordingly, it can be advantageous to ensure that that cold front 307 does not impinge on the nerve 326.

As mentioned above, some tissue, like nerve tissue, may be transiently affected prior to being irreversibly damaged. More particularly, the ability of certain nerve tissue to conduct impulses to muscles may be affected by temperatures that are warmer than those temperatures that cause permanent nerve damage. For example, nerve tissue may be transiently affected at about 0° C. (that is, at about 0° C., the nerve tissue may temporarily stop conducting nerve impulses); whereas temperatures above 10° C. (but near or below normal body temperature) may have no effect on the nerve tissue's ability to conduct nerve impulses. Thus, in the scenario depicted in FIG. 3D, where the tissue structure 326 is the phrenic nerve, nerve impulses may be blocked in the section of the nerve 326 impinged upon by the cooler region 323. Because the impulses of the left or right phrenic nerve control the corresponding left or right side of one's diaphragm, blocking such impulses in one of the phrenic nerves can impact diaphragm function and overall respiration. In particular, tidal volume may be reduced, airflow or pressure in the airway may be reduced, overall blood oxygenation may be reduced, and respiratory rate may be increased, and any of these effects can be readily detected by the sensors 141A, 141B or 141C, and controller 133 described above.

By indirectly detecting that the cooler region 323 has impinged upon the phrenic nerve 326, and at that point stopping or suspending delivery of cryotherapy, the controller 133 can prevent the cold front 307 from reaching the phrenic nerve and causing permanent damage (as is depicted in FIG. 3E). Moreover, automating this process with sensors and the controller 133 may be a more reliable and safer way of protecting a patient's phrenic nerve than other methods of detecting a transient effect on the phrenic nerve. One such other method may include, for example, pacing the phrenic nerve from the coronary sinus. A physician may place one of his or her hands over the right upper diaphragm to feel twitches of the diaphragm muscle (or more precisely, to feel twitches of the diaphragm muscle stop, indicating that the phrenic nerve has been at least temporarily affected). The above-described indirect detection method essentially uses the patient's brain for pacing, and regular respiration activity as the result of such “pacing,” rather than relying on electrical pacing and a phsyician's detection of corresponding pacing-induced muscle twitches. That is, function of the phrenic nerve may be monitored without artificial pacing, and any impact to phrenic nerve function may be readily and reliably detected.

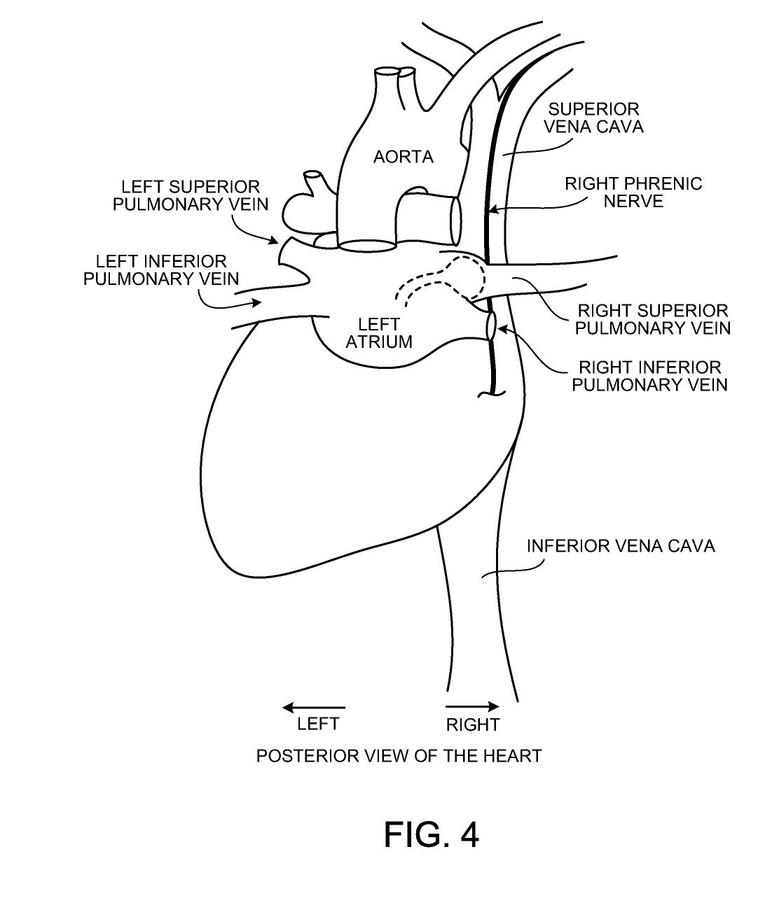

FIG. 4 illustrates the anatomical relationship between the pulmonary veins of a typical patient and the right phrenic nerve and provides additional context for the preceding description. In the context of treating atrial fibrillation, protecting the right phrenic nerve during treatment of the right superior pulmonary vein is particularly important, given the proximity of these structures.

FIG. 4 provides a posterior view (i.e., a view from the back) of a typical patient's heart. As shown, the left atrium is situated near the top of the back surface of the heart. Four pulmonary veins exit the back of the left atrium—two from each side. These pulmonary veins are designated as left or right pulmonary veins, and superior (top) or inferior (bottom) veins. The inferior vena cava runs up the right front side of the heart and meets the superior vena cava, which runs down the right front side of the heart. The right phrenic nerve typically follows the superior vena cava as shown, passing fairly closely to the right superior pulmonary vein, before running across pericardial tissue (not shown in FIG. 4), to the diaphragm (below the heart, but also not shown in FIG. 4). Thus, as depicted in FIG. 4, the right phrenic nerve typically comes the closest to the right superior pulmonary vein. Note that the left phrenic nerve, which is not shown in FIG. 4, does not generally come as close to any of the pulmonary veins as the right phrenic nerve does. Accordingly, protecting the right phrenic nerve during a cryotherapy procedure directed to isolating pulmonary veins is generally of greater concern than protecting the left phrenic nerve. However, the left phrenic nerve is typically situated close to the left atrial appendage (not shown), and thus, cryotherapy procedures directed to sites in or near the left atrial appendage may employ the systems and methods described in this document to protect the left phrenic nerve during such procedures.

Significant variation in distance between the right phrenic nerve and the right pulmonary veins (particularly the right superior pulmonary vein) has been observed. Accordingly, delivery of cryotherapy to the right superior pulmonary vein of some patients, even for long periods of time, may have little effect on those patients' right phrenic nerves. That is, the right phrenic nerve of such a patient may be disposed far enough from the outer wall of the right pulmonary vein that a cold front propagating from a balloon catheter inside the left atrium, at the ostium of the right superior pulmonary vein, may not ever reach the right phrenic nerve. On the other hand, the right phrenic nerves in other patients may be very close to those patients' right superior pulmonary veins, such that delivery of cryotherapy to the right superior pulmonary veins may pose significant risk to these patients.

Because it is not always possible to determine a precise distance between the phrenic nerve and the pulmonary veins, a cryotherapy system that automatically detects physiological conditions (e.g., changes in respiration function that may result from transient impairment of the diaphragm) that likely correspond to the phrenic nerve being chilled, and suspends delivery of the cryotherapy upon detection of such conditions, can facilitate a safer cryotherapy procedure. That is, although other methods may enable detection of whether the phrenic nerve is affected by delivery of cryotherapy (e.g., pacing the phrenic nerve and manually or tactilely monitoring the effect of the pacing), electronically monitoring respiration parameters and automatically suspending the delivery of cryotherapy can provide another layer of safety to a cryotherapy procedure.

In some implementations, for example in order to balance safety and procedure efficacy, a second threshold may be employed to provide a warning, prior to automatically suspending delivery of cryotherapy. For example, if a change in a respiration parameter of 25% or more of the baseline value is detected, the system may automatically suspend delivery of cryotherapy. It may be advantageous, however, to provide a warning signal when a change of 10-15% is detected. With such a warning, a physician who is delivering the cryotherapy may be made aware of the possible risk, and may also be able to take steps to reduce the risk and still complete the procedure in a manner that is likely to treat the underlying condition. More specifically, for example, after receiving a warning indicator (e.g., an audible alarm or a visual indicator), a physician may reduce the rate at which cryotherapy is delivered. In some procedures, reducing the rate may facilitate continued therapeutic cooling of the targeted tissue, while reducing the depth to which a cold front associated with the cooling penetrates beyond the targeted tissue—which may have the effect of protecting the phrenic nerve while allowing the procedure to proceed for a longer period of time than might otherwise be possible, absent the warning signal.

Additional countermeasures may be taken to protect the phrenic nerve. For example, particularly in cases in which it is determined that the phrenic nerve is very close to a pulmonary vein being treated, heat may be applied internal to the patient to slow propagation of the cold front 307 beyond the pulmonary vein. More particularly, a heater (e.g., an infrared or other radiant heater, or another type of heater) may be disposed in the patient's esophagus, as close as possible to a region of the pulmonary vein being treated and the phrenic nerve being protected—to counteract the cooling effect on the phrenic nerve of the cryotherapy and possibly extending the time during which cryotherapy can be applied to the pulmonary vein.

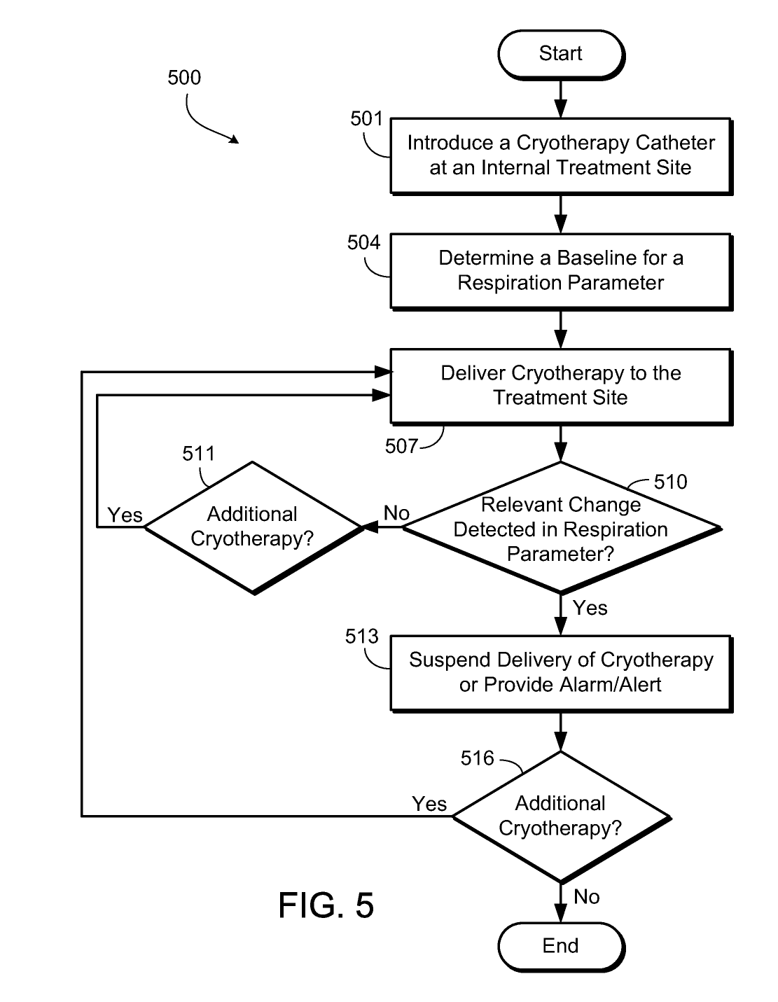

FIG. 5 is a flow diagram of an example method 500 of providing cryotherapy. In some implementations, the method is performed by a system such as the system 50 shown in FIG. 1. The method 500 can include introducing (501) a cryotherapy catheter at a treatment site inside a patient's heart. For example, the catheter 100 can be advanced through a patient's vasculature and into the left atrium 268, and the treatment component of the catheter (e.g., the balloon 103) can be positioned against an ostium or antrum of one of the pulmonary veins (e.g., the right superior pulmonary vein).

The method 500 can include determining (504) a baseline for a respiration parameter of the patient. For example, the system 50 can employ an extensiometer 141A to measure a baseline chest or abdomen expansion associated with normal breathing. More particularly, the system 50 can employ a signal processor 142 to analyze peaks associated with chest expansion (or more precisely, resistance or some other electrical parameter that varies as the patient's chest expands and contracts). The analyzed peaks may be stored as data values 144 that correspond to one or more respiration cycle amplitudes.

The method 500 can include delivering (507) cryotherapy to a treatment site of the patient. That is, the controller 133 can regulate valves 136 and 139 to control the flow of a cryogenic agent to and from the balloon 103, in order to ablate tissue at the treatment site. While the cryotherapy is being delivered, the method 500 can include monitoring (510) the respiration parameter of the patient. As long as the respiration parameter does not change, relative to the baseline, by more than a threshold value, cryotherapy can continue to be delivered (507) according to an appropriate treatment protocol. As depicted, a determination (511) can be made as to whether additional cryotherapy is called for by the treatment protocol.

During delivery of cryotherapy, the system 50 can continue to employ the extensiometer 141A to monitor chest expansion and contraction. A signal processor 142 can analyze the data from the sensor 141A in real-time, or substantially real-time, to identify peaks in the real-time signal, correlate the real-time peaks to peaks in the baseline, and determine whether differences between the two exceed a threshold. If a change between the respiration parameter and the baseline that exceeds the threshold value is detected, then an alarm can be enabled or delivery of the cryotherapy can be suspended (513). In FIG. 1, two respiration cycles are depicted (i.e., the first two of four cycles depicted) in which there is little variation between baseline data 144 and real-time data 147; two additional respiration cycles are depicted (i.e., the third and fourth of four cycles) in which differences between baseline and real-time data exceed a threshold. In physiological terms, the third and four cycles depict reduced chest or abdomen expansion, which may be caused by transient paralysis of a portion of the patient's diaphragm (which may, in turn, be caused by chilling of the phrenic nerve caused by delivery of cryotherapy to a nearby treatment site). Suspending the delivery of cryotherapy upon detection of a change relative to the baseline (e.g., the change 149 in FIG. 1) may protect the patient from irreversible nerve damage, as described above.

After delivery of cryotherapy is completed at one treatment site (or the delivery of cryotherapy is suspended because of a detected change in the respiration parameter), additional cryotherapy can be delivered (516). For example, in some implementations, additional cryotherapy is delivered to the same treatment site, after the tissue has warmed up. (In some implementations, additional cryotherapy may not be delivered to a treatment site once the delivery of cryotherapy has been suspended, given the high risk to permanent nerve damage that additional cryotherapy may pose.) In other implementations, delivery of additional cryotherapy can include delivery of cryotherapy to a different treatment site. In particular, for example, each of four different pulmonary veins may be treated, and after one pulmonary vein is treated, the catheter may be moved to the antrum or ostium of a different pulmonary vein, at which additional cryotherapy can be delivered.

A number of implementations have been described. Nevertheless, it will be understood that various modifications may be made without departing from the spirit and scope of this document. For example, the systems and methods described herein can be applied in procedures directed to treating conditions other than atrial fibrillation. Modes of cooling, other than evaporation of refrigerant, can be employed. In particular, a cryogenic agent can be employed that remains in either a liquid or gas state. Moreover, the methods and systems described herein can be employed in RF ablation systems to detect transient effects on nerves during an RF ablation procedure and gate the delivery of additional RF energy or provide a warning or alarm. The systems and methods described herein can be extended to protect nerves other than the phrenic nerve, and other physiological processes can be monitored (e.g., processes other than respiration) to track a state of the different nerve(s) to be protected. Accordingly, other implementations are within the scope of the following claims.

What is claimed is:

1. A cryotherapy delivery system, the system comprising:
a cryotherapy catheter having a distal treatment component that delivers, during a cryotherapy procedure, cryotherapy to a treatment site inside a patient's body;
a controller that controls the delivery of the cryotherapy during the cryotherapy procedure; and
a sensor that measures values of a respiration parameter of the patient during the cryotherapy procedure, and provides measured values to the controller;
wherein the controller a) determines a baseline value for the respiration parameter; b) detects, during delivery of the cryotherapy, a change in the respiration parameter relative to the baseline value; and c) suspends delivery of the cryotherapy when the change exceeds a threshold.

2. The cryotherapy delivery system of claim 1, wherein the controller controls the delivery of the cryotherapy by regulating the flow of a cryogenic agent to and from the distal treatment component to regulate a temperature of the treatment component.

3. The cryotherapy delivery system of claim 1, wherein the controller provides an alarm signal when the change exceeds a warning threshold that is smaller than the threshold.

4. The cryotherapy delivery system of claim 3, wherein the warning threshold is approximately 10%.

5. The cryotherapy delivery system of claim 1, wherein the threshold is approximately 25%.

6. The cryotherapy delivery system of claim 1, wherein the sensor comprises an extensiometer that measures expansion and contraction of the patient's chest or abdomen.

7. The cryotherapy delivery system of claim 1, wherein the sensor comprises an air flow monitor or tidal volume monitor that measures an inspiratory flow rate or expiratory flow rate.

8. The cryotherapy delivery system of claim 1, wherein the sensor comprises a pulse oximeter that measure an oxygen saturation value of the patient's blood.

9. The cryotherapy delivery system of claim 1, wherein the treatment component comprises an expandable balloon.

10. A method of providing cryotherapy, the method comprising:
introducing a cryotherapy catheter at a treatment site inside a patient's heart;
determining a baseline value for a respiration parameter of the patient;
employing an electronic controller of the cryotherapy catheter to regulate delivery of cryotherapy to the treatment site;
while cryotherapy is being delivered to the treatment site, detecting a change in the respiration parameter, relative to the baseline value, that exceeds a threshold;
in response to detecting the change, employing the electronic controller to automatically suspend delivery of the cryotherapy.

11. The method of claim 10, wherein the threshold is approximately 50% of the average baseline value.

12. The method of claim 10, wherein the treatment site is an antrum or ostium of a pulmonary vein of the patient.

13. The method of claim 10, wherein detecting a change that exceeds the threshold comprises detecting a change in function of the patient's diaphragm that is indicative of transient paralysis of the patient's phrenic nerve.

14. The method of claim 10, wherein determining the baseline and detecting the change comprise receiving values from a sensor that is coupled to the electronic controller.

15. The method of claim 14, wherein the sensor comprises an extensiometer that measures expansion and contraction of the patient's chest or abdomen.

16. The method of claim 14, wherein the sensor comprises a flow monitor that measures an inspiratory flow rate, expiratory flow rate or tidal volume.

17. The method of claim 14, wherein the sensor comprises a pulse oximeter that measures an oxygen saturation value of the patient's blood.

18. The method of claim 14, wherein the sensor measures values corresponding to the patient's chest expansion and respiratory frequency.

19. The method of claim 10, further comprising supplying heat to a region of the patient's esophagus that is in close proximity to the treatment site.

20. The method of claim 14, wherein the sensor comprises an impedance plethysmography that measures changes in chest impedance.
